(19) United States
(12) Patent Application Publication
(10) Pub. No.: US 2025/0359991 A1
(43) Pub. Date: Nov. 27, 2025

(54) DELIVERY DEVICE HAVING STABILITY TUBE WITH COMPRESSIBLE REGION

(71) Applicant: Medtronic, Inc., Minneapolis, MN (US)

(72) Inventor: Jake Dunlea, Galway (IE)

(21) Appl. No.: 18/872,181

(22) PCT Filed: Jun. 13, 2023
(86) PCT No.: PCT/IB2023/056082

§ 371 Date: Dec. 5, 2024

Related U.S. Application Data
(60) Provisional application No. 63/352,837, filed on Jun. 16, 2022.

(51) Int. Cl. A61F 2/24

(52) U.S. Cl. CPC A61F 2/2436

(57) ABSTRACT

Transcatheter prosthetic heart valve delivery devices including an inner shaft assembly having a coupling structure configured to selectively engage a prosthetic heart valve, a delivery sheath assembly having a capsule and slidably disposed over the inner shaft assembly, and an outer stability tube coaxially received over the delivery sheath assembly and having a compressible region. As the capsule is retracted, the capsule contacts a distal end of the outer stability tube and, with further proximal retraction of the capsule, force is applied by capsule to the distal end of the outer stability tube and eventually overcomes a biasing force of the compressible region, which causes the outer stability tube to retract along with the capsule allowing the outer stability tube to be longer and provider greater stability than if the compressible region were not present.

### FIELD

The present technology is generally related to transcatheter prosthetic heart valve delivery devices.

### BACKGROUND

A human heart includes four heart valves that define the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of a prosthetic heart valve or prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during percutaneous transluminal delivery, and expanded upon deployment at or within the native valve. One type of valve stent can be initially provided in an expanded or uncrimped arrangement, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath for transluminal delivery. Retracting the sheath from this valved stent allows the stent to self-expand to a larger diameter, fixating at the native valve site. Once the prosthetic valve is positioned at the treatment site, for instance within an incompetent native valve, the stent frame structure may be expanded to hold the prosthetic valve firmly in place.

The present disclosure addresses problems and limitations associated with the related art.

### SUMMARY

The techniques of this disclosure generally relate to transcatheter valve delivery/deployment systems and devices including an outer stability tube. Such delivery devices generally include a handle assembly, delivery sheath assembly, inner shaft assembly and outer stability tube. The delivery sheath assembly includes a capsule for sheathing a prosthetic heart valve maintained on the inner shaft assembly during delivery. In a delivery state, the prosthetic heart valve is compressed over the inner shaft and within the capsule. The outer stability tube is disposed over the delivery sheath assembly. The outer stability tube isolates the delivery sheath assembly from an introducer at the distalmost end of the delivery device and stabilizes the inner shaft assembly and the delivery sheath assembly while anchoring the delivery device relative to the patient anatomy when the delivery sheath assembly is retracted to unsheathe and deploy the prosthetic heart valve.

The outer stability tube is sized so that its distal end is proximally spaced from the capsule (in the delivery state) by a distance approximating a length of the capsule itself. This allows for full retraction of the delivery sheath assembly and the capsule to release and deploy the prosthetic heart valve. Improved device stability during deployment of the prosthetic heart valve may be provided if the outer stability tube is extended closer to the capsule (e.g., if the outer stability tube is sized so that its distal end is proximally spaced from the capsule (in the delivery state) by a distance approximating less than a length of the capsule itself). Therefore, aspects of the disclosure include providing an outer stability tube with a compressible region that is biased to an uncompressed state. With this approach, a distal end of the outer stability tube can be positioned close to or adjacent the capsule in the delivery state. As the capsule is retracted, the capsule contacts the distal end of the outer stability tube and, with further proximal retraction of the capsule, force is applied by the capsule to the distal end of the outer stability tube and eventually overcomes a biasing force of the compressible region, which causes the outer stability tube to move proximally and retract along with the capsule.

In one aspect, the present disclosure provides a transcatheter prosthetic heart valve delivery device including an inner shaft assembly having a coupling structure configured to selectively engage a prosthetic heart valve, a delivery sheath assembly slidably disposed over the inner shaft assembly, and an outer stability tube coaxially received over the delivery sheath assembly and has a first portion and a compressible region. The compressible region has an axial compressive strength that is less than an axial compressive strength of the first portion. Devices further include a handle assembly maintaining the inner shaft assembly, the delivery sheath assembly, and the outer stability tube. The handle assembly includes a housing secured to the inner shaft assembly and an actuator mechanism maintained by the housing and coupled to the delivery sheath assembly. The actuator mechanism is operable to selectively move the delivery sheath assembly relative to the inner shaft assembly.

In another aspect, the disclosure provides a transcatheter prosthetic heart valve delivery device including an inner shaft assembly configured to selectively engage a prosthetic heart valve, an outer stability tube, and a handle assembly maintaining the inner shaft assembly and the outer stability tube. The handle assembly has a housing secured to the inner shaft assembly and a biasing member connecting the outer stability tube with the housing.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a partially exploded, perspective view of a delivery device for delivering a prosthetic heart valve including an outer stability tube having a compressible region within a handle assembly.

FIG. 2A is a cross-sectional view of the delivery device of FIG. 1 in a delivery state having the prosthetic heart valve omitted for clarity.

FIG. 2B is a cross-sectional view of the delivery device of FIG. 2A in a deployment state having the prosthetic heart valve omitted for clarity.

FIG. 3 is a cross-sectional view of an alternate delivery device for delivering a prosthetic heart valve including an outer stability tube having a compressible region adjacent the handle assembly.

FIG. 4 is a cross-sectional view of yet another delivery device for delivering a prosthetic heart valve including an outer stability tube having a compressible region between two sheath portions of the outer stability tube.

### DETAILED DESCRIPTION

Specific embodiments of the present disclosure are now described with reference to the figures, where like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

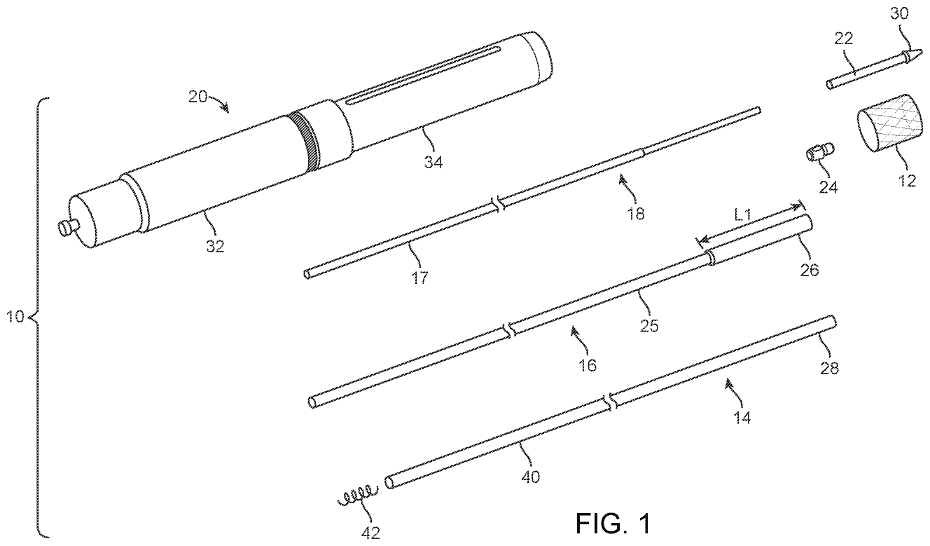

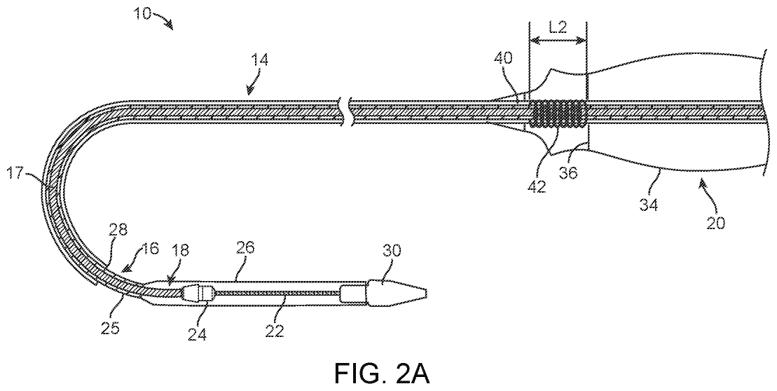

Components of one non-limiting example of a delivery device 10 are illustrated in FIGS. 1-2B. The delivery device 10 is arranged and configured for percutaneously delivering a cardiac prosthesis 12, such as a stented prosthetic heart valve or stent. With reference to FIG. 1 which shows an exploded perspective view, the delivery device 10 includes an outer stability tube assembly 14, a delivery sheath assembly 16, an inner shaft assembly 18, and a handle assembly 20.

The inner shaft assembly 18 includes a proximal inner shaft 17 and a distal shaft 22 having a nose cone 30 disposed at a distal end of the distal inner shaft 22. The nose cone 30 may be conically shaped or otherwise adapted to promote atraumatic contact with bodily tissue. The distal inner shaft 22 may be continuous with the proximal inner shaft 17 or may optionally be coupled to the proximal inner shaft 17 with a retainer 24. For example, retainer 24 couples a proximal end of the distal inner shaft 22 to a distal end of the proximal inner shaft 17. In addition to coupling the proximal inner shaft 17 and the distal inner shaft 22, the retainer 24 may have one or more recesses oriented to retain one or more protrusions of the prosthesis 12 to secure the prosthesis 12 to the inner shaft assembly 18 in a radially compressed configuration for percutaneous delivery to a treatment site (e.g., a patient's heart).

The delivery sheath assembly 16 includes a delivery tube 25 and a capsule 26 disposed at a distal end of the delivery tube 25. The capsule 26 extends distally from the delivery tube 25 a distance L1 and has an internal volume oriented to encapsulate the prosthesis 12 in the radially compressed configuration for percutaneous delivery to the treatment site. The outer stability tube assembly 14 includes an outer stability tube 40 having a distal end 28 and a compressible member 42. The inner shaft assembly 18, delivery sheath assembly 16, and outer stability tube assembly 14 are sized to be concentrically arranged, with the proximal inner shaft 17 positioned concentrically within the delivery tube 25, and the delivery tube 25 positioned concentrically within the outer stability tube 40. Any one or more of the outer stability tube assembly 14, delivery sheath assembly 16, and inner shaft assembly 18 may be a singular, integrally formed component or can alternatively include multiple interconnected components.

The handle assembly 20 includes a housing 34 and an actuator 32 that is configured to move one or more of the inner shaft assembly 18, delivery sheath assembly 16, and outer stability tube assembly 14. For example, the actuator 32 may be configured to move the delivery sheath assembly 16 proximally and/or distally relative to the inner shaft assembly 18. Movement of the delivery sheath assembly 16 proximally relative to the inner shaft assembly 18 proximally moves the capsule 26 relative to the distal inner shaft 22 thereby unsheathing the radially compressed prosthesis 12. As the prosthesis 12 is unsheathed by the proximally moving capsule 26, the prosthesis 12 radially expands until the entire prosthesis 12 is unsheathed and deployed at the treatment site. Movement of the delivery sheath assembly 16 distally relative to the inner shaft assembly 18 distally moves the capsule 26 relative to the distal inner shaft 22 thereby re-sheathing or recapturing radially expanded portions of the prosthesis 12.

As shown in FIGS. 2A and 2B, which illustrate a cross-sectional view of the delivery device 10 in a delivery state (FIG. 2A) and a deployment state (FIG. 2B), the proximal inner shaft 17 of the inner shaft assembly 18 extends distally from the handle assembly 20 axially along and inside of a delivery tube 25 of the delivery sheath assembly 16. Similarly, the delivery tube 25 extends distally from the handle assembly 20 axially along and inside of the outer stability tube 40 of the outer stability tube assembly 14. The capsule 26 extends distally from the delivery tube 25 along the distal inner shaft 22 towards the nose cone 30 and encapsulates the compressed prosthesis 12 (not shown) and the retainer 24. A proximal end of the capsule 26 is axially spaced from the distal end 28 of the outer stability tube 40.

Once loaded, compressed, and covered by the capsule 26 of the delivery sheath assembly 16 in the delivery state, the prosthetic heart valve 12 is delivered to the target site, such as a native heart valve. During deployment of the prosthetic heart valve 12 at the target site, the outer stability tube 40 improves stability of the delivery sheath assembly 16 and the inner shaft assembly 18 by resisting the tendency for these components to move from the outer to the inner curvature of the anatomy (e.g., when traversing along the aortic arch). When the prosthetic heart valve 12 is at the target site, the delivery sheath assembly 16 and the capsule 26 are proximally withdrawn with respect to the distal inner shaft portion 22 of the inner shaft assembly 18 and the prosthetic heart valve 12 loaded thereto such that a proximal end of the capsule 26 contacts the distal end 28 of the outer stability tube assembly 14. When the capsule 26 is proximally withdrawn in a deployment state, the prosthetic heart valve 12 is allowed to expand to an expanded arrangement, partially releasing and ultimately fully deploying the prosthetic heart valve 12 from the distal inner shaft portion 22 and the retainer 24 of the inner shaft assembly 18. . . . In some embodiments, the prosthetic heart valve 12 can be expanded using any known technique such as with an expandable balloon or naturally if the prosthetic heart valve 12 is made of a shape memory material configured to be biased to the expanded arrangement. Should recapture of the prosthetic heart valve 12 be desired for either repositioning of the prosthesis or bailout of the procedure, the capsule 26 can be distally advanced prior to full deployment of the prosthesis.

Additionally, in the deployment state, proximal retraction of the delivery sheath assembly 16 relative to the handle assembly 20 applies a longitudinal force onto the outer stability tube assembly 14 at an interface between the capsule 26 and the distal end 28 of the outer stability tube 40. Distal and proximal movement of the delivery sheath assembly 16 including the delivery tube 25 and the capsule 26 relative to the prosthetic heart valve 12 can be actuated by the handle assembly 20. The handle assembly 20 can take many configurations for both supporting the delivery sheath assembly 16 and the inner shaft assembly 18 as well as controlling movement of each of these components. In one example, the handle assembly 20 can include an actuator mechanism 32 that is rotatable such that rotational movement of the actuator mechanism 32 about a central longitudinal axis of the handle assembly 20 corresponds to translational movement of the delivery sheath assembly 16 to move the capsule 26 relative to the inner shaft assembly 18 to either sheathe or unsheathe the prosthetic heart valve 12. Any other actuator mechanism capable of controlling the movement of the capsule 26 are considered within the scope of the present disclosure.

In one example, the compressible region or member 42 of the outer stability tube assembly 14 is positioned within a housing 34 of the handle assembly 20 such that the compressible region 42 abuts an interior surface 36 of the housing 34 (which can, in some examples, be considered a component within the housing 34). The compressible region 42 can provide a biasing force to urge a distal end 28 of the outer stability tube 40 to a distalmost position during delivery of the prosthetic heart valve 12 and can also compress against the interior surface 36 to reduce its length. For example, FIG. 2A shows the compressible region 42 having a first length L2 in a relatively uncompressed configuration thereby biasing the outer stability tube 40 in a distal direction toward the capsule 26 such that the distal end 28 of the outer stability tube 40 is at a distalmost position during delivery of the prosthetic heart valve 12. Conversely, in FIG. 2B, upon proximal retraction of the delivery sheath assembly 16, the capsule 26 abuts the distal end 28 of the outer stability tube 40 thereby exerting an axial compressive force on the outer stability tube assembly 14 causing the compressible region 42 to compress to a shorter, second length L3. The compressible region 42 of the outer stability tube assembly 14 has a relatively lower axial compressive strength compared to a relatively higher axial compressive strength of other portions of the outer stability tube 40. In some examples, the compressible region 42 has a lower axial compressive strength compare to adjacent portions of the outer stability tube 40. Thus, when subjected to an axial compressive force imparted by the proximal retraction of the delivery sheath assembly 16 that is greater than the biasing force of the compressive region 42, the compressive region 42 is configured to compress and reduce in length from L2 to L3.

In some embodiments, the outer stability tube 40 may be sized so that the distal end 28 is proximally spaced from the capsule 26 (in the delivery state) by a distance approximating a longitudinal length L1 of the capsule 26 itself or greater to ensure that the capsule 26 is not impeded from full retraction by the outer stability tube assembly 14. This sizing allows for full retraction of the delivery sheath assembly 16 and capsule 26 to release and deploy the prosthetic heart valve 12. During prosthesis deployment, improved stability is provided when the outer stability tube 40 extends closer to the capsule 26 and a distance between the distal end 28 of the outer stability tube 40 and the capsule 26 is less than the length L1 of the capsule 26). Thus, compared to prior embodiments where the distance between the distal end 28 of the outer stability tube 40 and the capsule 26 are approximately equal to or greater than the length L1 of the capsule 26, embodiments of the disclosure include increasing a length of the outer stability tube 40 so that the distal end 28 of the outer stability tube 40 is closer to the capsule 26 at a predetermined distance defined as less than the length L1 of the capsule 26. As the capsule 26 is retracted for deployment of the prosthetic heart valve 12, the capsule 26 contacts the distal end 28 of the outer stability tube 40 and, with further proximal retraction of the capsule 26, force is applied by capsule 26 to the distal end 28 of the outer stability tube 40. This force eventually overcomes the biasing force of the compressible region 42, allowing the outer stability tube 40 to retract in conjunction with the capsule 26. The handle assembly 20 can be configured to permit proximal retraction of the outer stability tube 40 relative to the handle assembly 20 when a compressive force applied to the outer stability tube 40 overcomes a biasing force of the compressible region 42. For example, by spacing the distal end 28 of the outer stability tube 40 at a distance from the capsule 26 that is less than the length LI of the capsule 26, proximal retraction of the delivery sheath assembly 16 relative to the inner shaft assembly 18 applies the force onto the outer stability tube 40 that overcomes the biasing force of the compressible region 42.

The compressible region 42 of embodiments disclosed herein can take many forms. In the illustrated example, the compressible region 42 is formed at least in part by a biasing member such as a compression spring that is connected to the outer stability tube 40. The compressible region 42 may optionally be hollow, tubular, or otherwise define a longitudinal opening through which other components of the delivery device 10 (e.g., one or more of the delivery tube 25, the inner shaft 17, a guide wire (not shown), and one or more flush lumens (not shown) can be positioned and/or move through. In other examples, the compressible region 42 could be manufactured from one or more materials capable of exerting a biasing force such as a foam or other resilient, compressible material. Some non-limiting examples of the compressible material include a polymer, foam, rubber, braid/coil construction, laser cut hypotube, or other structures having compressible geometry, all of which have a compressive strength less than a comparable compressive strength of the outer stability tube 40 such that the compressible region 42 is configured to compress prior to the outer stability tube 40 under an applied compressive load.

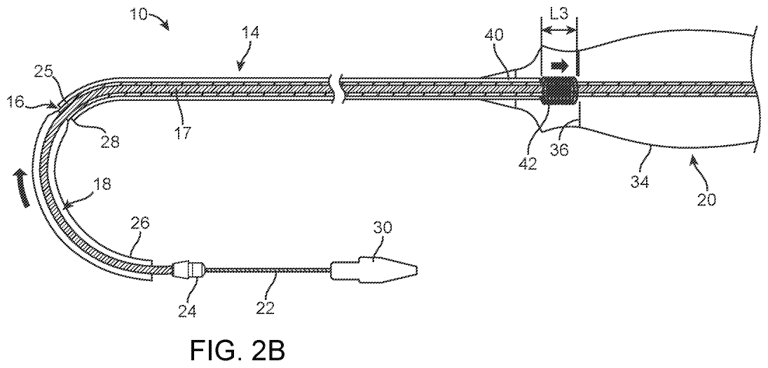

FIG. 3 illustrates a cross-sectional view of another delivery device 110 that is the same as or similar to the delivery device 10 of FIGS. 1-2B, except as otherwise stated. In this example, the delivery device 110 includes an outer stability tube assembly 114 coaxially positioned over the delivery sheath assembly 16 and having a compressible region 142 and an outer stability tube 140. The compressible region 142 is positioned at least partially, if not entirely, outside of the housing 34 of the handle assembly 20. In this example, the compressible region 142 is positioned to abut an exterior surface 136 of the housing 34 so that the compressible region 142 compresses against the exterior surface 136 of the housing 34 either directly or indirectly. For example, in some embodiments, the compressible region 142 may compress directly against the exterior surface 136 but, in other examples, an unspecified element may be positioned between the exterior surface 136 and the compressible region 142. The compressible region 142 can take any form disclosed herein. It is further envisioned that the compressible region 142 can optionally include a covering 144 to prevent potential pinching of the anatomy or operation materials including, but not limited to, surgical drapes, operator gloves, or the like. In some examples, the compressible region 142 has an outer diameter that is equal to an outer diameter of the outer stability tube 140 to provide a substantially seamless transition from the outer stability tube 140 to the compressible region 142 in the delivery state (i.e. when the compressible region 142 is not compressed).

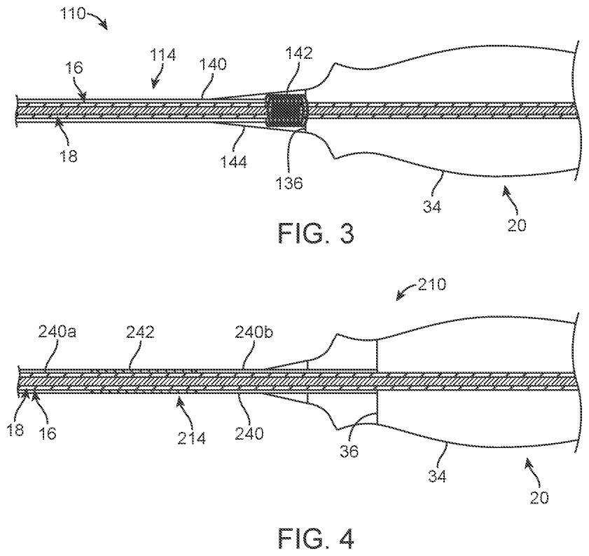

FIG. 4 illustrates a cross-sectional view of another alternate delivery device 210 that is the same as or similar to the delivery device 10 of FIGS. 1-2B, except as otherwise stated. In this example, the delivery device 210 includes an outer stability tube assembly 214 having a compressible region 242. The compressible region 242 is positioned entirely outside of the housing 34 of the handle assembly 20 and between a distal portion 240a of an outer stability tube 240 and a proximal portion 240b of the outer stability tube 240. The compressible region 242 may optionally be positioned such that it is located within the patient during delivery of the prosthetic heart valve 12. The proximal portion 240b of the outer stability tube 240 can have sufficient rigidity along its length to allow the compressible region 242 to effectively compress against the proximal portion 240b without the proximal sheath portion 240b compressing. Alternatively, the proximal portion 240b can be configured to have varying rigidity from the compressible region 242 to the handle assembly 20. For example, the proximal sheath portion 240b can have a compressive stiffness that increases in a direction defined from the compressible region 242 toward the handle assembly 20, thereby providing a variable stiffness that allows the compressible region 242 and a portion of the proximal sheath portion 240b to compress under an applied load. The compressible region 242 can take any form disclosed herein and can optionally include a covering (e.g., covering 144) as disclosed above. In some examples, the compressible region 242 has an outer diameter that is seamless and equal to an outer diameter of the distal portion 240a and/or the proximal portion 240b of the outer stability tube 240. The outer stability tube assembly 214 can be a sheath having a plurality of interconnected portions having variable compressibility. The outer stability tube assembly 214 can be made of one or more materials to provide the variance in compressibility and the biasing force at one or more regions.

As referred to herein, prosthetic heart valves useful with the various devices and methods of the present disclosure may assume a wide variety of configurations, such as a bioprosthetic heart valve having tissue leaflets or a synthetic heart valve having polymeric, metallic, or tissue-engineered leaflets, and can be specifically configured for replacing valves of the human heart. The prosthetic heart valves of the present disclosure may be self-expandable, balloon expandable, and/or mechanically expandable or combinations thereof. The prosthetic heart valves of the present disclosure include a stent or stent frame having an internal lumen maintaining a valve structure (tissue or synthetic), with the stent frame having a normal, expanded condition or arrangement and collapsible to a compressed condition or arrangement for loading within the delivery device. For example, the stents or stent frames are support structures that include a number of struts or wire segments arranged relative to each other to provide a desired compressibility and strength to the prosthetic heart valve. The struts or wire segments are arranged such that they are capable of self-transitioning from, or being forced from, a compressed or collapsed condition to a normal, radially expanded condition. The struts or wire segments can be formed from a shape memory material, such as a nickel titanium alloy (e.g., nitinol). The stent frame can be laser-cut from a single piece of material, or can be assembled from a number of discrete components.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A transcatheter prosthetic heart valve delivery device comprising:
an inner shaft assembly including a coupling structure configured to selectively engage a prosthetic heart valve;
a delivery sheath assembly slidably disposed over the inner shaft assembly;
an outer stability tube coaxially received over the delivery sheath assembly and having a first portion and a compressible region; wherein the compressible region has an axial compressive strength that is less than an axial compressive strength of the first portion; and
a handle assembly maintaining the inner shaft assembly, the delivery sheath assembly, and the outer stability tube, the handle assembly including:
a housing secured to the inner shaft assembly, and
an actuator mechanism maintained by the housing and coupled to the delivery sheath assembly, wherein the actuator mechanism is operable to selectively move the delivery sheath assembly relative to the inner shaft assembly.

2. The transcatheter prosthetic heart valve delivery device of claim 1, wherein the compressible region includes a biasing member.

3. The transcatheter prosthetic heart valve delivery device of claim 2, wherein the handle assembly is configured to permit proximal retraction of the outer stability tube relative to the housing when a force applied to the outer stability tube overcomes a force of the biasing member.

4. The transcatheter prosthetic heart valve delivery device of claim 3, wherein the handle assembly is configured such that the biasing member applies the force onto the outer stability tube to resist movement of the outer stability tube relative to the housing.

5. The transcatheter prosthetic heart valve delivery device of claim 3, wherein the transcatheter prosthetic heart valve delivery device is configured such that proximal retraction of the delivery sheath assembly relative to the inner shaft assembly applies the force onto the outer stability tube.

6. The transcatheter prosthetic heart valve delivery device of claim 5, wherein the delivery sheath assembly includes a proximal shaft and a distal capsule configured to contain the prosthetic heart valve, and further wherein the transcatheter prosthetic heart valve delivery device is configured to provide:
a delivery state in which the capsule is distally spaced from a distal end of the outer stability tube, and
a deployment state in which the capsule is proximally retracted from the delivery state and contacts the distal end of the outer stability tube.

7. The transcatheter prosthetic heart valve delivery device of claim 6, wherein the transcatheter prosthetic heart valve delivery device is configured such that in the deployment state, proximal retraction of the delivery sheath assembly relative to the handle applies a longitudinal force onto the outer stability tube at an interface between the capsule and the distal end.

8. The transcatheter prosthetic heart valve delivery device of claim 1, wherein the compressible region abuts an exterior surface of the housing.

9. The transcatheter prosthetic heart valve delivery device of claim 1, wherein the compressible region is positioned within the housing.

10. The transcatheter prosthetic heart valve delivery device of claim 1, wherein the compressible region includes a spring.

11. A transcatheter prosthetic heart valve delivery device comprising:
an inner shaft assembly configured to selectively engage a prosthetic heart valve;
an outer stability tube; and
a handle assembly maintaining the inner shaft assembly and the outer stability tube, the handle assembly including:
a housing secured to the inner shaft assembly,
a biasing member connecting the outer stability tube with the housing.

12. The transcatheter prosthetic heart valve delivery device of claim 11, wherein the handle assembly is configured to permit proximal retraction of the outer stability tube relative to the housing when a force applied to the outer stability tube overcomes a force of the biasing member.

13. The transcatheter prosthetic heart valve delivery device of claim 12, wherein the handle assembly is configured such that the biasing member applies the force onto the outer stability tube to resist movement of the outer stability tube relative to the housing.

14. The transcatheter prosthetic heart valve delivery device of claim 12, further comprising a delivery sheath assembly; wherein the transcatheter prosthetic heart valve delivery device is configured such that proximal retraction of the delivery sheath assembly relative to the inner shaft assembly applies the force onto the outer stability tube.

15. The transcatheter prosthetic heart valve delivery device of claim 14, wherein the delivery sheath assembly includes a proximal shaft and a distal capsule configured to contain the prosthetic heart valve, and further wherein the transcatheter prosthetic heart valve delivery device is configured to provide:
a delivery state in which the capsule is distally spaced from a distal end of the outer stability tube, and
a deployment state in which the capsule is proximally retracted from the delivery state and contacts the distal end of the outer stability tube.

16. The transcatheter prosthetic heart valve delivery device of claim 15, wherein the transcatheter prosthetic heart valve delivery device is configured such that in the deployment state, proximal retraction of the delivery sheath assembly relative to the handle applies a longitudinal force onto the outer stability tube at an interface between the capsule and the distal end.

17. The transcatheter prosthetic heart valve delivery device of claim 11, wherein the biasing member includes a spring.

18. The transcatheter prosthetic heart valve delivery device of claim 11, wherein the handle assembly includes an actuator mechanism maintained by the housing and coupled to the delivery sheath assembly, wherein the actuator mechanism is operable to selectively move the delivery sheath assembly relative to the inner shaft assembly.

19. The transcatheter prosthetic heart valve delivery device of claim 11, wherein the inner shaft includes a coupling structure configured to selectively engage the prosthetic heart valve.

20. The transcatheter prosthetic heart valve delivery device of claim 11, further comprising a delivery sheath assembly slidably disposed over the inner shaft assembly and at least partially within the outer stability tube.
